(19) United States
(12) Patent Application Publication
(10) Pub. No.: US 2012/0041541 A1
(43) Pub. Date: Feb. 16, 2012

(54) IMPLANT AND METHOD FOR THE PRODUCTION THEREOF

(75) Inventors: Ullrich Bayer, Admannshagen-Bargeshagen (DE); Felix Baader, Eglisau (CH); Eric Wittchow, Nuernberg (DE)

(73) Assignee: BIOTRONIK AG, Buelach (CH)

(21) Appl. No.: 13/206,204

(22) Filed: Aug. 9, 2011

Related U.S. Application Data
(60) Provisional application No. 61/373,283, filed on Aug. 13, 2010.

(51) Int. Cl. A61F 2/82; C25D 11/02; C25D 5/54; C25D 5/44; C25D 11/04

(52) U.S. Cl. 623/1.15; 216/37; 205/324; 205/159; 205/325

(57) ABSTRACT

An implant, in particular an intraluminal endoprosthesis, having an implant body (2, 4) containing an aluminum compound, preferably an aluminum alloy and/or aluminum oxide. Improved biocompatibility of the implant is achieved in that at least the part of the surface of the implant body (2, 4) which is formed by the aluminum compound has a first layer (6) which contains an aluminum phosphate. Furthermore, a cost-effective method for producing such an implant is described.

### CROSS REFERENCE TO RELATED APPLICATIONS

This invention claims priority to U.S. patent application Ser. No. 61/373,283 filed Aug. 13, 2010; the contents of which are herein incorporated by reference in their entirety.

### TECHNICAL FIELD

The present invention relates to a method for producing an implant, in particular an intraluminal endoprosthesis.

### BACKGROUND

Medical endoprostheses or implants for greatly varying applications are known in a great manifold from the prior art. Implants in the meaning of the present invention are to be understood as endovascular prostheses or other endoprostheses, such as stents, orthopedic implants such as fasteners for bones, such as screws, plates, or nails, surgical suture material, intestinal clamps, vascular clips, prostheses in the area of the hard and soft tissue, and anchor elements for electrodes, in particular of pacemakers or defibrillators.

Currently, stents are particularly frequently employed as implants, which are used for the treatment of stenoses (vascular constrictions). They have a body in the form of a possibly pierced tubular or hollow-cylindrical main lattice, which is open at both longitudinal ends. The implant body of such an endoprosthesis is inserted into the vessel to be treated and is used for the purpose of supporting the vessel. Stents have established themselves in particular for the treatment of vascular illnesses. Constricted areas in the vessels may be expanded by the use of stents, so that an increase of lumen results. Through the use of stents or other implants, an optimum vascular cross-section, which is primarily required for the treatment success, can be achieved, however, the permanent presence of a foreign body of this type initiates a cascade of microbiological processes, which may result in gradual ingrowth of the stent and in vascular closure in the worst case. An approach for solving this problem comprises manufacturing the stent or other implants from a biodegradable material.

Biodegradation is understood to mean hydrolytic, enzymatic, or other metabolically-related degradation processes in the living organism, which are caused above all by the bodily fluids coming into contact with the endoprosthesis and result in gradual dissolving of at least large parts of the endoprosthesis. The implant loses its mechanical integrity at a specific time through this process. The term biocorrosion is frequently used synonymously with the term biodegradation. The term bioresorption comprises the subsequent resorption of the degradation products by the living organism.

Materials suitable for the body of biodegradable implants may contain polymers or metals, for example. The body can comprise a plurality of these materials. The common feature of these materials is their biodegradability. Examples of suitable polymer compounds are polymers from the group cellulose, collagen, albumin, casein, polysaccharides (PSAC), polylactide (PLA), poly-L-lactide (PLLA), polyglycol (PGA), poly-D,L-lactide-co-glycolide (PDLLA-PGA), polyhydroxy butyric acid (PHB), polyhydroxy valeric acid (PHV), polyalkyl carbonates, polyorthoester, polyethylene terephtalate (PET), polymalonic acid (PML), polyanhydrides, polyphosphazenes, polyamino acids, and their copolymers, and hyaluronic acid. Depending on the desired properties, the polymers may be provided in pure form, in derivative form, in the form of blends, or as copolymers. Metal biodegradable materials are predominantly based on alloys of magnesium, aluminum, and iron. The present invention preferably relates to implants whose biodegradable material of the implant body at least partially contains aluminum, in particular an aluminum-based alloy (in short hereafter: aluminum alloy) and/or aluminum oxide.

When implementing biodegradable implants, an effort is made to control the degradability in accordance with the desired therapy or the application of the respective implant (coronary, intracranial, renal, etc.). For example, it is an important target range for many therapeutic applications that the implant loses its integrity in a period of time from four weeks to six months. Integrity, i.e., mechanical integrity, is understood here as the property that the implant hardly has mechanical damage in relation to the non-degraded implant. This means that the implant is still so mechanically stable that, for example, the collapse pressure has dropped only slightly, i.e., at most to 80% of the nominal value. The implant can thus still fulfill its main function, ensuring the ability to pass the vessel, with existing integrity. Alternatively, the integrity can be defined to mean that the implant is still so mechanically stable that it is hardly subjected to geometrical changes in its load state in the vessel, for example, it does not noticeably crumple, i.e., it has at least 80% of the dilation diameter under load, or it hardly has any broken load-bearing struts in the case of a stent.

Implants having an iron alloy, in particular ferrous stents, are particularly cost-effective and simple to produce. For example, for the treatment of stenoses, these implants lose their mechanical integrity or support action only after a comparatively long period of time, i.e., only after a dwell time in the treated organism of approximately 2 years. This means that the collapse pressure in the case of ferrous implants is reduced too slowly over time for the desired applications.

In contrast, implants having a magnesium alloy frequently degrade too rapidly. Magnesium implants may additionally be deformed very poorly.

A further possibility comprises manufacturing implants predominantly from an aluminum alloy. The advantage of the use of aluminum alloys for implants is that they have significantly better deformation capability because of their face-centered cubic crystal lattice in comparison to the magnesium alloys having hexagonal structure. Aluminum alloys have a higher modulus of elasticity in comparison to magnesium alloys (approximately 70 GPa in relation to approximately 45 GPa for a magnesium alloy). The good mechanical properties of the aluminum alloy are useful in particular in the case of stent dilation or during production of the semi-finished product, which can then be performed more cost-effectively. A medical implant which comprises an alloy having the main component aluminum is mentioned in the publication DE 197 31 021 A1.

However, the biocompatibility of this material is discussed controversially in connection with aluminum implants, in particular with respect to vascular inflammation and the probability of necrosis.

### DETAILED DESCRIPTION

Therefore, the object of the present invention is to specify a cost-effective method for producing an implant, which causes a degradation of the implant in the desired time window. Furthermore, the implant produced according to the method is to have good biocompatibility. Correspondingly, the object of the invention additionally comprises providing such an implant.

The object stated above is achieved by an implant in which at least the part of the surface of the implant body which is formed by the aluminum compound has a first layer, which contains an aluminum phosphate.

In the present invention, the body of the implant comprises at least a part of the implant, preferably the main part of the implant, which causes the mechanical integrity of the implant.

The at least one first layer made of the aluminum phosphate (AlPO₄), which is implemented as a monolayer, shields the endothelial cells of the body directly after the implantation of the implant from the metal aluminum of the aluminum compound, so that tissue inflammation can be very extensively suppressed. This results in a high tissue compatibility, which prevents cell reactions in particular in the first minutes and hours after the positioning of the implant in the body environment.

In the further proceedings, infiltration of the thin layer made of aluminum phosphate, which has a thickness of approximately 0.5 nm to approximately 10 nm, preferably approximately 1 nm to approximately 5 nm in a preferred exemplary embodiment, occurs under body milieu conditions. During the infiltration of the first layer, aluminum hydroxides are formed. The formation of hydroxides additionally causes a slow weakening of the implant integrity by degradation.

The inventors have recognized that initially a contact between metal aluminum and the tissue is avoided using an aluminum phosphate layer. Through the initially beginning degradation of the non-metal surface, the adjoining tissue only comes into contact slowly with metal aluminum. Necrosis and a suddenly occurring high concentration of free aluminum ions are thus avoided.

In a refinement of the invention, a second layer containing an acid-degradable polymer, preferably PLLA and/or PLGA, which preferably contains a pharmaceutically active substance, is additionally provided on the first layer. Accordingly, in particular a polylactide, a polyglycoside, or a copolymer thereof, particularly preferably PLLA or PLGA, or a blend of the mentioned polymers is used as the acid-degradable polymer. The reaction speed of the hydroxides, which form during the infiltration of the first layer made of the aluminum phosphate, can vary through the presence of the acid-degradable polymers.

For this purpose, a “pharmaceutically active substance” (or therapeutically active or effective substance) is understood in the meaning of the invention as a vegetable, animal, or synthetic agent (medication) or a hormone, which is used in suitable dosage as a therapeutic agent for influencing states or functions of the body, as a replacement for natural agents produced by the human or animal body, such as insulin, and for removing or making harmless pathogens, tumors, cancer cells, or body foreign materials. The release of the substance in the environment of the implant has a positive effect on the course of healing or counteracts pathological changes of the tissue as a result of the surgical intervention and/or is used for making malignant cells harmless in oncology

Such pharmaceutically active substances have, for example, an anti-inflammatory and/or anti-proliferative and/or spasmolytic effect, whereby, for example, restenosis, inflammations, or (vascular) spasms may be prevented. Such substances may comprise one or more substances of the agent group of calcium channel blockers, lipid regulators (such as fibrates), immunosuppressive agents, calcineurin inhibitors (such as tacrolimus), antiphlogistic agents (such as cortisone or diclofenac), anti-inflammatory agents (such as imidazoles), anti-allergy agents, oligonucleotides (such as dODN), estrogens (such as genistein), endothelial formers (such as fibrin), steroids, proteins, hormones, insulins, cytostatic agents, peptides, vasodilators (such as sartane), and materials having an antiproliferative effect, taxols or taxanes, preferably paclitaxel or sirolimus here.

The above-described hydroxides additionally have the advantage that they act as an adjuvant when pharmaceutically active substances (agent carriers) are intercalated in the second layer (also referred to as a topcoat). In this way, they may be influenced in their solution behavior or in their elution rate as a function of the indication.

The implant according to the invention has a degradation speed in the range of 1 to 2 years.

A degradation speed elevated in relation thereto is achieved in that the aluminum compound is an aluminum alloy having alloy elements from the group containing copper and silver. The alloy elements copper, silver, and indium are preferably intercalated at the grain boundaries of the microstructure and thus accelerate the inter-crystalline corrosion. Upon the presence of the specified alloy elements, the integral dwell time in the organism of the implant according to the invention thus drops in comparison to implants made of an aluminum alloy without the preferred alloy elements. The mentioned alloy elements additionally have cytostatic or cytotoxic effects and thus prevent proliferation.

The mentioned alloy elements additionally form inter-metallic compounds when the solubility limit is exceeded, which also act in the grain interior as local elements and displace the position of the bulk material in the electrochemical series toward less noble values.

Furthermore, the alloy elements copper, silver, and calcium result in a higher degradation speed, because they interfere with the mechanisms which form the corrosion-protecting passive layer made of aluminum oxide. In addition, the corrosion which progresses in the bulk material is accelerated by formation of local elements.

A further advantage of the alloy elements from the group containing copper and silver is that these alloy elements cause a mixed crystal solidification, which results in an increase of the yield strength and the tensile strength. In this way, the mechanical properties of the implant are to improved and a reduction of the dimensions of the supporting structures of the implant (such as the reduction of the diameter of a stent strut) is made possible. A material savings can also be achieved in this way.

In a preferred exemplary embodiment, the body of the implant predominantly contains aluminum, in particular more than 80 wt.-% aluminum, particularly preferably at least 95 wt.-% aluminum, in particular in an alloy. These alloys have the above-specified degradation time.

Furthermore, the above stated object is achieved by a method comprising the following steps:
a) providing the implant body,
b) applying a first layer containing an aluminum phosphate to at least a part of the surface of the implant body, which is formed by the aluminum compound, preferably using phosphoric acid anodization.

Using a method of this type, an improvement of the biocompatibility is achieved very cost-effectively. In particular the treatment of the implant body using phosphoric acid anodization for producing the aluminum phosphate layer causes the formation of micro-cavities, which allow optimum adhesion of a further layer. The relatively large pores (mean diameter of the pores of approximately 50 nm) and the rough outer surface, in particular in the case of the formation of the aluminum compound as an aluminum oxide, promote the mechanical inter-locking of the structures of the aluminum oxide with a further layer situated over it.

In a further exemplary embodiment, a second layer containing an acid-degradable polymer, such as a polylactide, a polyglycoside, or a copolymer thereof, particularly preferably PLLA or PLGA, or a blend of the mentioned polymers, can be applied on the first layer made of aluminum phosphate after step b). This polymer can additionally contain a pharmaceutically active substance. Such a second layer has the above-specified advantages.

Such a second coating is preferably produced using spraying or using immersion in a diluted polymer solution. In the latter possibility, the solvent is subsequently thermally expelled.

Furthermore, it is advantageous if the implant body is provided with a high degree of strain hardening before step b) and/or an age hardening is performed before step b) at a temperature is of 130° C. to 180° C. over a period of time of at least 60 minutes to 48 hours. The high degree of strain hardening and the final tempering treatment prevent growth of the grains of the aluminum alloy by recrystallization. Inter-crystalline corrosion is encouraged in this way.

It is also advantageous for the application of a further coating if the implant body is etched before step b) using a base, preferably NaOH and/or KOH. The etching is preferably performed at a temperature of approximately 50° C. Approximately 4 μm deep pores are generated in this way, which cause a microscopic pushbutton effect with the further layer. In a preferred embodiment step, the etching of the pores is terminated by pickling of the implant body in 15% HNO₃. This treatment flushes away the alloy elements exposed by the etching and leaves behind a blank metal surface.

The above-stated object is further achieved by an implant obtainable by an above-described method according to the invention. Such an implant has the above advantages specified in connection with the production method according to the invention.

The method according to the invention and the implant according to the invention are explained hereafter on the basis of examples and a single FIGURE. All features shown or described form the subject matter of the invention, independently of their combination in the claims or to what they refer.

### DESCRIPTION OF THE DRAWING

In the FIGURE:

FIG. 1 is a schematic showing a cross-section through a superficial volume of an implant according to the invention, which is implemented as a stent.

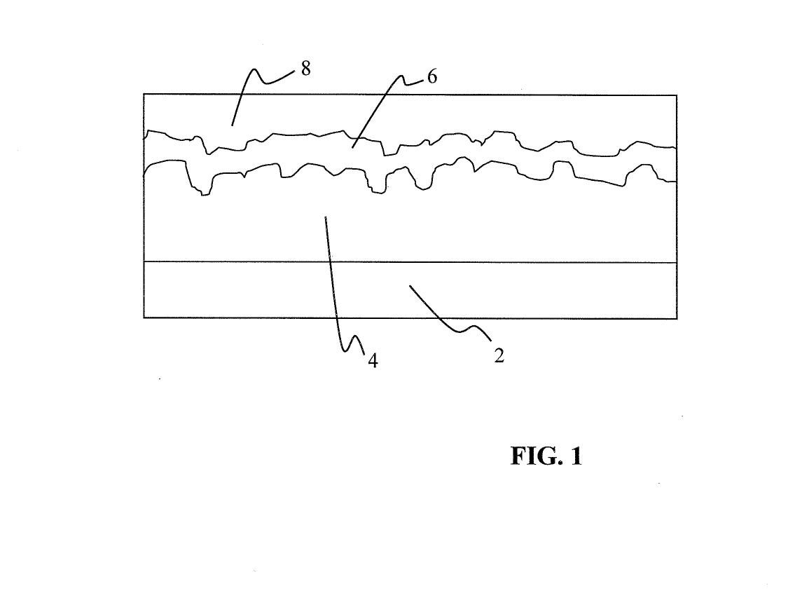

### DETAILED DESCRIPTION

### First Example

The body 2 of a stent comprises an aluminum alloy having (specifications in wt.-%) 4% Cu, 2% Ag, 0.5% Ca. The tube dimensions of the stent body are approximately 2 mm for the external diameter and approximately 140 μm for the wall thickness. The aluminum alloy has a tensile strength of >350 MPa, a yield strength of >250 MPa at >12% ultimate elongation and a degree of cold deformation >30%. A layer 4 made of aluminum oxide, which is approximately 2 nm to 4 nm thick, is formed on the surface of the stent body 2 by the oxidation processes frequently occurring during the treatment and storage.

The material was subjected to a thermal age hardening at a temperature of approximately 150° C. over a period of time of approximately one hour.

A stent body 2 having the lattice structure explained in the introduction to the description is produced from a block or billet made of the specified aluminum alloy using known methods (laser cutting up to electropolishing). The stent body 2 is subsequently etched in aqueous NaOH/KOH solution (10 vol.-% per component each) at 50° C. The stent body thus treated is then flushed over 2 minutes twice at 50° C. in running H₂O.

The stent is now pickled in 15 vol.-% HNO₃ over a period of time of 20 seconds and room temperature. The component is also moved. Flushing is subsequently performed twice in H₂O at 50° C. with ultrasonic support. The surface of the stent body has a jagged structure having an array of pores after the etching and pickling, which allows good fastening of the first layer 6, which is now produced, containing the aluminum compound.

The phosphoric acid anodization to produce the first layer 6 containing an aluminum phosphate is performed in 8% H₃PO₄ over a period of time of 20 minutes and at an anodization voltage of 14 V. In this way, an approximately 2 nm thick AlPO₄ layer 6 is produced. Subsequently, the coated stent body is flushed again twice at 50° C. in H₂O. The component is now dried.

Finally, the coated stent body is sprayed using a degradable polymer charged with agent, such as PLLA L210, using known methods. The second layer 8 results, which has a thickness of 1 to 2 μm. The second layer 8 is also referred to as a topcoat

### Second Example

A stent body 2 comprising an aluminum alloy having 3% copper, 3% silver, 0.2% calcium, and 0.1% indium (specifications each in wt.-%) is used and is subjected to a phosphoric acid anodization to produce the first layer 6 containing an aluminum phosphate. For this purpose, the stent body is immersed in 100% H₃PO₄ and treated over a period of time of 10 minutes and at an anodization voltage of 14 V. In this way, an approximately 2 nm thick AlPO₄ layer 6 is produced. The coated stent body 2 is subsequently flushed twice at 50° C. in H₂O. The coated component is now dried.

In addition, the pretreatment and posttreatment steps specified in the first example may be performed.

### Third Example

An alloy as specified in the first example is used for the stent body 2 and a phosphoric acid anodization as described in the second example is performed. The stent body 2 has an external diameter of approximately 1.8 mm and a wall thickness of approximately 110 μm.

The pretreatment and posttreatment steps specified in the first example may also be performed in this example.

### Fourth Example

The third example corresponds to the first example up to the application of the topcoat 8. In the last step, a degradable polymer blend (such as PLGA 85/15) charged with an agent (such as Paclitaxel) is sprayed on in such a manner that a second layer 8 having a layer thickness of approximately 1 μm to 2 μm results.

It will be apparent to those skilled in the art that numerous modifications and variations of the described examples and embodiments are possible in light of the above teaching. The disclosed examples and embodiments are presented for purposes of illustration only. Therefore, it is the intent to cover all such modifications and alternate embodiments as may come within the true scope of this invention.

### LIST OF REFERENCE NUMERALS

2 stent body made of an aluminum alloy
4 aluminum oxide layer, which is also a component of the stent body
6 first layer
8 second layer

What is claimed is:

1. An implant, in particular intra-luminal endoprosthesis, having an implant body containing an aluminum compound, optionally an aluminum alloy and/or aluminum oxide, wherein at least a part of a surface of the implant body which is formed by the aluminum compound has a first layer, which contains an aluminum phosphate.

2. The implant according to claim 1, characterized in that the first layer has a thickness of approximately 0.5 nm to approximately 10 nm, optionally approximately 1 nm to approximately 5 nm.

3. The implant according to claim 1, characterized in that the aluminum compound is an aluminum alloy having alloy elements selected from the group containing copper and silver.

4. The implant according to claim 3, characterized in that the aluminum compound has additional alloy elements selected from the group containing calcium and indium.

5. The implant according to claim 1, characterized in that the implant body contains more than 80 wt.-% aluminum, optionally at least 95 wt.-% aluminum.

6. The implant according to claim 1, characterized in that a second layer contains an acid-degradable polymer, optionally PLLA and/or PLGA, which optionally contains a pharmaceutically active substance is additionally provided on the first layer.

7. A method for producing an implant, in particular an intraluminal endoprosthesis, having an implant body containing an aluminum compound, preferably an aluminum alloy and/or aluminum oxide, comprising the following steps:
a) providing the implant body,
b) applying a first layer containing an aluminum phosphate to at least a part of the surface of the implant body which is formed by the aluminum compound, optionally using phosphoric acid anodization.

8. The method according to claim 7, characterized in that the aluminum compound is an aluminum alloy having alloy elements selected from the group containing copper and silver, and optionally having additional alloy elements selected from the group containing calcium and Indian.

9. The method according to claim 7, characterized in that the implant body is provided with a high degree of strain hardening before step b) and/or an age hardening is performed at a temperature in the range from 130° C. to 180° C. over a period of time from at least approximately 60 minutes to at most approximately 48 hours.

10. The method according to claim 7, characterized in that the implant body is etched using a basic solution, optionally NaOH and/or KOH, before step b).

11. The method according to claim 7, characterized in that a second layer containing an acid-degradable polymer, which optionally contains a pharmaceutically active substance, is additionally applied, optionally using spraying, to the implant body having the first layer.

12. An implant, in particular intraluminal endoprosthesis, having a body containing an aluminum compound, optionally an aluminum alloy and/or aluminum oxide, obtainable by a method according to claim 7.
